(19) United States
(12) Patent Application Publication
(10) Pub. No.: US 2024/0335641 A1
(43) Pub. Date: Oct. 10, 2024

(54) PASSIVE SAFETY CATHETER SYSTEM AND RELATED METHODS

(71) Applicant: Becton, Dickinson and Company, Franklin Lakes, NJ (US)

(72) Inventors: Edwin Jeyaseelan A., Madurai, Tamil Nadu (IN); Balaji Kannan, Bangalore, Karnataka (IN); Mukilan Balamurugan, Theni, Tamil Nadu (IN); Darin Charles Vincent, Salt Lake City, UT (US)

(21) Appl. No.: 18/618,940

(22) Filed: Mar. 27, 2024

Related U.S. Application Data
(60) Provisional application No. 63/457,641, filed on Apr. 6, 2023.

(51) Int. Cl. A61M 25/06; A61M 25/00

(52) U.S. Cl. CPC A61M 25/0631; A61M 25/0043; A61M 25/0097; A61M 25/0606

(57) ABSTRACT

A catheter system may include a catheter hub, which may include a distal end, a proximal end, and a lumen extending through the distal end of the catheter hub and the proximal end of the catheter hub. The catheter system may include a catheter extending from the distal end of the catheter hub. The catheter system may include a needle hub and an introducer needle extending from the needle hub. The introducer needle may extend through the catheter when the introducer needle is in an advanced position. The catheter system may include a compressible element or another needle shield element configured to shield the introducer needle from distal movement beyond the compressible element or the other needle shield element.

### RELATED APPLICATIONS

This application claims the benefit of U.S. Provisional Patent Application No. 63/457,641, which was filed on Apr. 6, 2023, which is incorporated herein in its entirety.

### BACKGROUND

A common type of catheter assembly includes a peripheral intravenous catheter (“PIVC”) that is over-the-needle. As its name implies, the PIVC that is over-the-needle may be mounted over an introducer needle having a sharp distal tip. The catheter assembly may include a catheter adapter, the PIVC extending distally from the catheter adapter, and the introducer needle extending through the PIVC. The PIVC and the introducer needle may be assembled such that the distal tip of the introducer needle extends beyond the distal tip of the PIVC with the bevel of the needle facing up away from skin of the patient immediately prior to insertion into the skin. The PIVC and the introducer needle are generally inserted at a shallow angle through the skin into vasculature of the patient.

In order to verify proper placement of the introducer needle and/or the PIVC in the blood vessel, a clinician may confirm that there is flashback of blood in a flashback chamber of the catheter assembly or in a space between the introducer needle and the PIVC. Once placement of the introducer needle has been confirmed, the clinician may remove the introducer needle, leaving the PIVC in place for future blood withdrawal or fluid infusion.

In some instances, the catheter assembly, such as, for example, the BD INSYTE™ AUTOGUARD™ Shielded IV Catheter, available from Becton Dickinson and Company of Franklin Lakes, New Jersey, may include a push button configured to retract the introducer needle after the PIVC is in place within the vein. A common problem with this type of retraction mechanism is blood splatter due to rapid and/or non-linear retraction of the introducer needle after depression of the push button. The blood splatter may contribute to increased occurrences of blood-borne virus exposures. Silicone gel is often used as a damper to delay a release time of a spring configured to retract the introducer needle, which may reduce the blood splatter. The silicone gel, however, may be messy and/or difficult to apply.

The subject matter claimed herein is not limited to embodiments that solve any disadvantages or that operate only in environments such as those described above. Rather, this background is only provided to illustrate one example technology area where some implementations described herein may be practiced.

### SUMMARY

The present disclosure relates generally to vascular access devices, systems, and methods. In particular, the present disclosure relates to a catheter system having a compressible element or other needle shield element configured to block or shield an introducer needle in a passive manner. Thus, the catheter system may block or shield the introducer needle automatically in response to proximal retraction of the introducer needle. In some embodiments, in addition to shielding the introducer needle and providing safety from needle stick injuries, the catheter system may also be configured to reduce blood splatter due to a reduced speed of the introducer needle during proximal retraction of the introducer needle compared to, for example, the BD INSYTE™ AUTOGUARD™ Shielded IV Catheter, available from Becton Dickinson and Company of Franklin Lakes, New Jersey.

In some embodiments, the catheter system may include a catheter hub, which may include a distal end, a proximal end, and a lumen extending through the distal end and the proximal end. In some embodiments, the catheter system may include a catheter extending from the distal end of the catheter hub. In some embodiments, the catheter system may include a needle hub and an introducer needle, which may extend distally from the needle hub. In some embodiments, the introducer needle may extend through the catheter when the introducer needle is in an advanced position.

In some embodiments, the catheter system may include a compressible element, which may include a first hole and a second hole. In some embodiments, the introducer needle may extend through the first hole and the second hole when the introducer needle is in the advanced position. In some embodiments, in response to movement of the introducer needle proximally to a retracted position, the introducer needle may be removed from the first hole and the second hole and the compressible element may expand. In some embodiments, in response to the compressible element expanding, the first hole and the second hole may misalign, shielding the introducer needle.

In some embodiments, the catheter system may include a housing. In some embodiments, the housing may include a pocket and an insertion slot transverse to a longitudinal axis of the housing and providing access to the insertion slot. In some embodiments, a distal end of the housing may be coupled to the proximal end of the catheter hub. In some embodiments, the compressible element may be disposed within the pocket. In some embodiments, the compressible element may be configured to compress in order to fit through the insertion slot and into the pocket.

In some embodiments, the housing may include an elongated slot aligned with the longitudinal axis of the housing. In some embodiments, a distal end of the housing may be coupled to the proximal end of the catheter hub. In some embodiments, the needle hub may extend through the elongated slot and may be configured to move along the elongated slot to move the introducer needle from the advanced position to the retracted position.

In some embodiments, the elongated slot may be a first elongated slot, and the housing may include a second elongated slot aligned with the longitudinal axis of the housing. In some embodiments, the needle hub may extend through the first elongated slot and may be configured to move along the first elongated slot and the second elongated slot to move the introducer needle from the advanced position to the retracted position. In some embodiments, a proximal end of the elongated slot may include a retainer snap.

In some embodiments, the compressible element may include a coil shape. In some embodiments, the first hole may be disposed in a first end of the coil shape and the second hole may be disposed in a second end of the coil shape. In some embodiments, the coil shape is flattened and/or may form a ring. In some embodiments, the first end of the coil shape and the second end of the coil shape may be positioned inwardly from the ring.

In some embodiments, the compressible element may include a first flange and a second flange. In some embodiments, the introducer needle may extend between the first flange and the second flange when the introducer needle is in the advanced position. In some embodiments, in response to movement of the introducer needle proximally to a retracted position, the introducer needle may not contact the first flange and the second flange and the first flange may overlap the second flange, shielding the introducer needle. In some embodiments, the compressible element may include the coil shape, which may be flattened and/or form a ring. In some embodiments, the first flange and the second flange may be positioned inwardly from the ring.

In some embodiments, the catheter system may include a needle shield element, which may include a movable center and multiple flexible arms extending outwardly from the movable center. In some embodiments, the movable center may include an aperture therethrough. In some embodiments, the introducer needle may extend through the aperture when the introducer needle is in the advanced position biasing the movable center in a first position. In some embodiments, in response to movement of the introducer needle proximally to a retracted position, the introducer needle may be removed from the aperture and the movable center may move to a second position in which the aperture is offset from a longitudinal axis of the introducer needle, shielding the introducer needle.

In some embodiments, the needle shield element may be disposed within the housing. In some embodiments, the needle hub may include a hub lock slot. In some embodiments, a proximal end of the housing may include a rib configured to lock within the hub lock slot in response to the introducer needle being moved from the advanced position to the retracted position.

It is to be understood that both the foregoing general description and the following detailed description are examples and explanatory and are not restrictive of the invention, as claimed. It should be understood that the various embodiments are not limited to the arrangements and instrumentality illustrated in the drawings. It should also be understood that the embodiments may be combined, or that other embodiments may be utilized and that structural changes, unless so claimed, may be made without departing from the scope of the various embodiments of the present invention. The following detailed description is, therefore, not to be taken in a limiting sense.

### BRIEF DESCRIPTION OF THE SEVERAL VIEWS OF THE DRAWINGS

Example embodiments will be described and explained with additional specificity and detail through the use of the accompanying drawings in which:

FIG. 1A is an upper perspective view of an example catheter system, illustrating an example introducer needle in an advanced position, according to some embodiments;

FIG. 1B is an upper perspective view of the catheter system, illustrating the introducer needle in an example retracted position, according to some embodiments;

FIG. 1C is a side view of the catheter system, illustrating the introducer needle in the advanced position, according to some embodiments;

FIG. 1D is a side view of the catheter system, illustrating the introducer needle in the retracted position, according to some embodiments;

FIG. 2A is a cross-sectional view of the catheter system along the line 2A-2A of FIG. 1C, according to some embodiments;

FIG. 2B is a cross-sectional view of the catheter system similar to the cross-sectional view of FIG. 2A but illustrating the introducer needle in between the advanced position and the retracted position, a portion of the introducer needle being shown for illustrative purposes, according to some embodiments;

FIG. 2C is a proximal-end view of an example compressible element, illustrating the compressible element in a compressed position, according to some embodiments;

FIG. 2D is an upper perspective view of the compressible element in the compressed position, according to some embodiments;

FIG. 2E is a side view of the compressible element, according to some embodiments;

FIG. 3A is a cross-sectional view of the catheter system along the line 3A-3A of FIG. 1D, according to some embodiments;

FIG. 3B is a cross-sectional view of the catheter system similar to the cross-sectional view of FIG. 3A but illustrating the introducer needle in the retracted position, a portion of the introducer needle being shown for illustrative purposes, according to some embodiments;

FIG. 3C is a proximal-end view of the compressible element, illustrating the compressible element in a relaxed position, according to some embodiments;

FIG. 3D is an upper perspective view of the compressible element, illustrating the compressible element in the relaxed position, according to some embodiments;

FIG. 4A is an upper perspective view of an example distal portion of an example housing, according to some embodiments;

FIG. 4B is an upper perspective view of the housing, according to some embodiments;

FIG. 4C is an upper perspective view of an example proximal end of the housing, according to some embodiments;

FIG. 4D is an upper perspective view of an example needle hub and example introducer needle extending from the needle hub, according to some embodiments;

FIG. 4E is an upper perspective view of an example catheter assembly, according to some embodiments;

FIG. 4F is a cross-sectional view of the catheter assembly, according to some embodiments;

FIG. 4G is a cross-sectional view of the catheter assembly, illustrating an example septum actuator coupled to a spring, according to some embodiments;

FIG. 5A is a cross-sectional view of the catheter system similar to the cross-sectional view of FIG. 2B but illustrating another compressible element, according to some embodiments;

FIG. 5B is an upper perspective view of the other compressible element, illustrating the compressible element in an example expanded position, according to some embodiments;

FIG. 5C is another upper perspective view of the other compressible element, illustrating the compressible element in the expanded position, according to some embodiments;

FIG. 5D is a cross-sectional view similar to FIG. 3B but illustrating the other compressible element in the expanded position, according to some embodiments;

FIG. 5E is an upper perspective view of the other compressible element in an example relaxed position, according to some embodiments;

FIG. 5F is another upper perspective view of the other compressible element in the relaxed position, according to some embodiments;

FIG. 6A is a partial cutaway view of the catheter system, illustrating the other compressible element in the relaxed position and the introducer needle in the retracted position, according to some embodiments;

FIG. 6B is a partial cutaway view of an example distal portion of the catheter system, illustrating the other compressible element in the expanded position and the introducer needle in the advanced position, according to some embodiments;

FIG. 6C is a partial cutaway view of the distal portion of the catheter system, illustrating the other compressible element in the relaxed position and the introducer needle in the retracted position, according to some embodiments;

FIG. 7A is a distal-end view of an example needle shield element, according to some embodiments;

FIG. 7B is an upper perspective view of the needle shield element, according to some embodiments;

FIG. 7C is a partial cutaway of the needle shield element disposed within the housing with a distal end of the housing removed for illustrative purposes and the needle in the advanced position, according to some embodiments;

FIG. 7D is a partial cutaway of the needle shield element disposed within the housing with a distal end of the housing removed for illustrative purposes and the needle in the retracted position, according to some embodiments;

FIG. 8A is an upper perspective view of an example housing having an example rib, according to some embodiments;

FIG. 8B is an upper perspective view of the needle hub configured to interact with the rib, according to some embodiments;

FIG. 8C is a cross-sectional view of the housing having the example rib coupled to the needle hub, according to some embodiments;

FIG. 8D is a bottom view of the housing having the example rib coupled to the needle hub, according to some embodiments;

FIG. 8E is a lower perspective view of the housing having the example rib coupled to the needle hub, according to some embodiments;

FIG. 9A is an upper perspective view of another example housing, according to some embodiments;

FIG. 9B is an upper perspective view of the other housing, according to some embodiments;

FIG. 9C is an upper perspective of a distal portion of the other housing coupled to the needle hub, according to some embodiments;

FIG. 9D is a lower perspective view of the other housing coupled to the catheter assembly, according to some embodiments;

FIG. 9E is a cross-sectional view of the other housing coupled to the catheter assembly along the line 9E-9E of FIG. 9D, according to some embodiments;

FIG. 9F is a side view of a catheter assembly including the other housing, illustrating the introducer needle in the advanced position, according to some embodiments;

FIG. 9G is a side view of the catheter assembly including the other housing, illustrating the introducer needle in the advanced position, according to some embodiments;

FIG. 9H is a cross-sectional view of the catheter assembly along the line 9H-9H of FIG. 9F, according to some embodiments; and

FIG. 9I is a cross-sectional view of the catheter assembly along the line 9I-91 of FIG. 9G, according to some embodiments.

### DESCRIPTION OF EMBODIMENTS

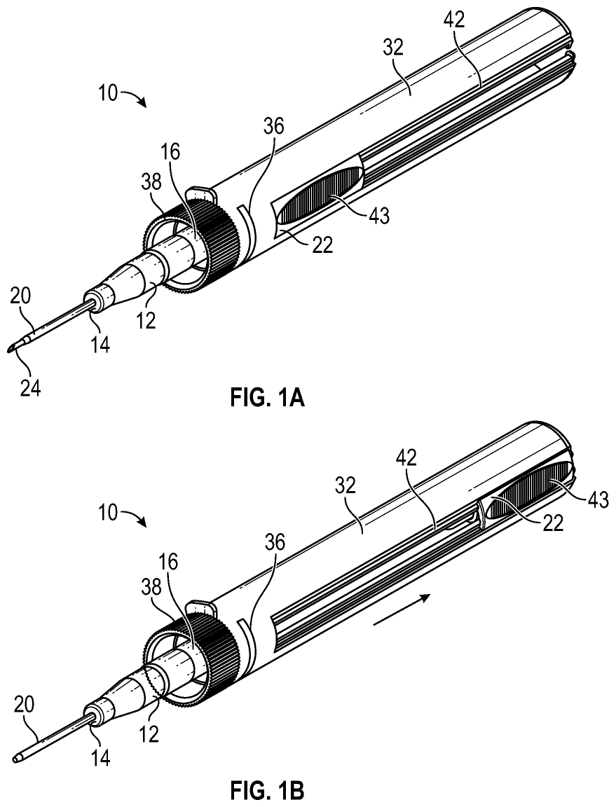

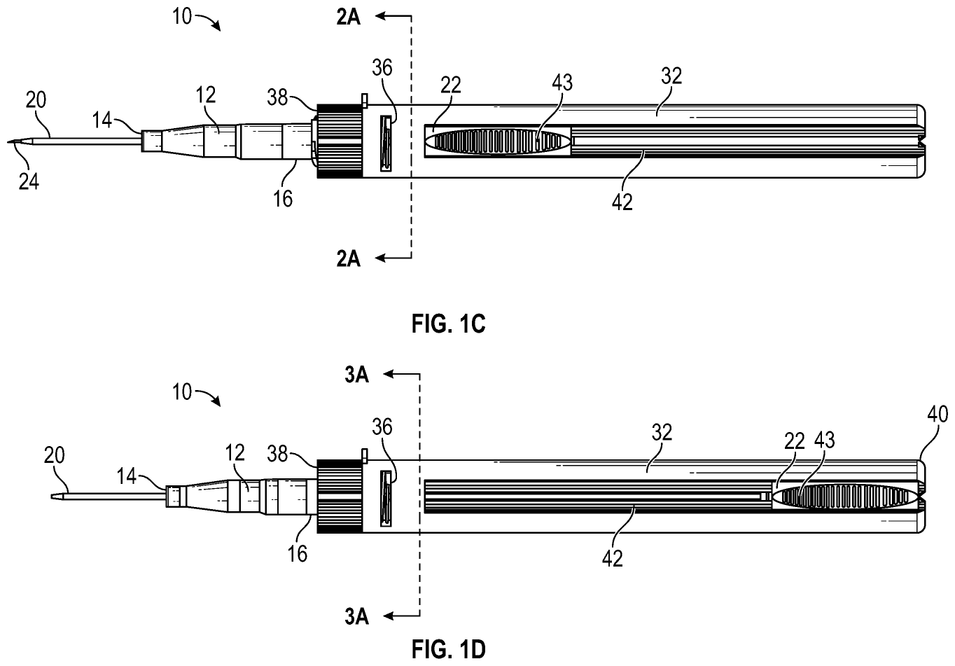

Referring now to FIGS. 1-3, a in some embodiments, a catheter system 10 may include a catheter hub 12, which may include a distal end 14, a proximal end 16, and a lumen 18 extending through the distal end 14 and the proximal end 16. In some embodiments, the catheter system 10 may include a catheter 20 extending from the distal end 14 of the catheter hub 12. In some embodiments, the catheter system 10 may include a needle hub 22 and an introducer needle 24, which may extend distally from the needle hub 22. In some embodiments, the introducer needle 24 may include a sharp distal tip configured to puncture through skin of a patient and facilitate insertion of the catheter 20 into a blood vessel of a patient. In some embodiments, the introducer needle 24 may extend through the catheter 20 when the introducer needle 24 is in an advanced position, as illustrated, for example, in FIGS. 1A and 1C.

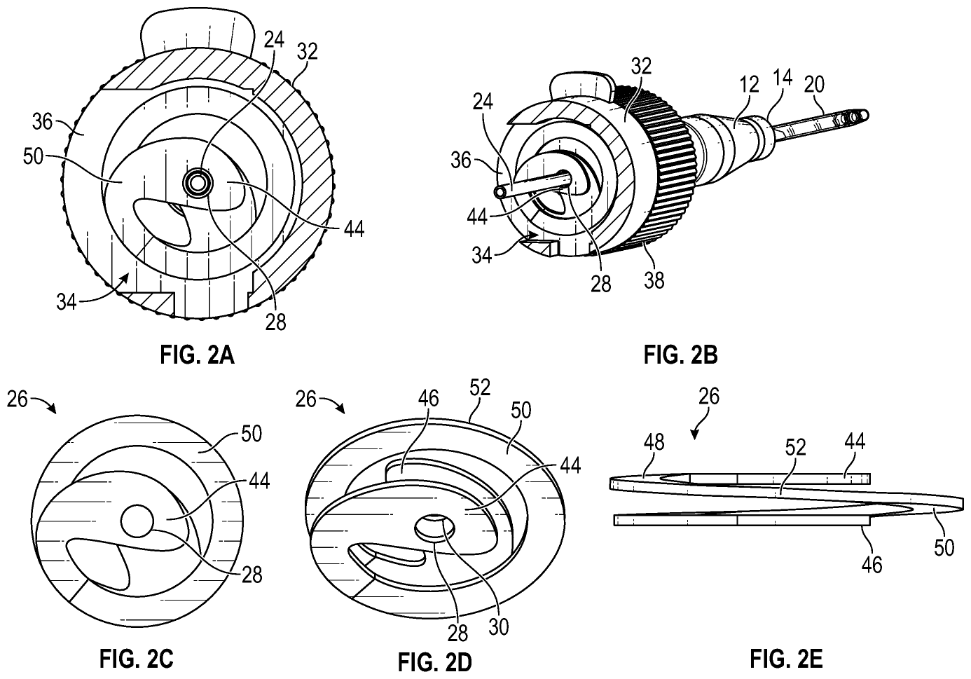

In some embodiments, the catheter system 10 may include a compressible element 26, which may include a first hole 28 and a second hole 30. In some embodiments, a size and a shape of the first hole 28 and the second hole 30 may be identical or similar. In some embodiments, a diameter of the first hole 28 and the second hole 30 may be slightly greater than an outer diameter of the introducer needle 24 such that the introducer needle 24 fits through the first hole 28 and the second hole 30. In some embodiments, the first hole 28 and/or the second hole 30 may be circular or a same shape as the introducer needle 24, which may facilitate less potential movement of the compressible element 26 when the introducer needle 24 extends through the first hole 28 and the second hole 30. In some embodiments, the introducer needle 24 may extend through the first hole 28 and the second hole 30 when the introducer needle 24 is in the advanced position, as illustrated, for example, in FIG. 2A. In these embodiments, the compressible element 26 may be in a compressed position. In some embodiments, the first hole 28 and the second hole 30 may be coaxial with the introducer needle 24 when the introducer needle 24 is in the advanced position. In some embodiments, in the advanced position, the first hole 28 may be aligned or concentrically aligned with the second hole 30.

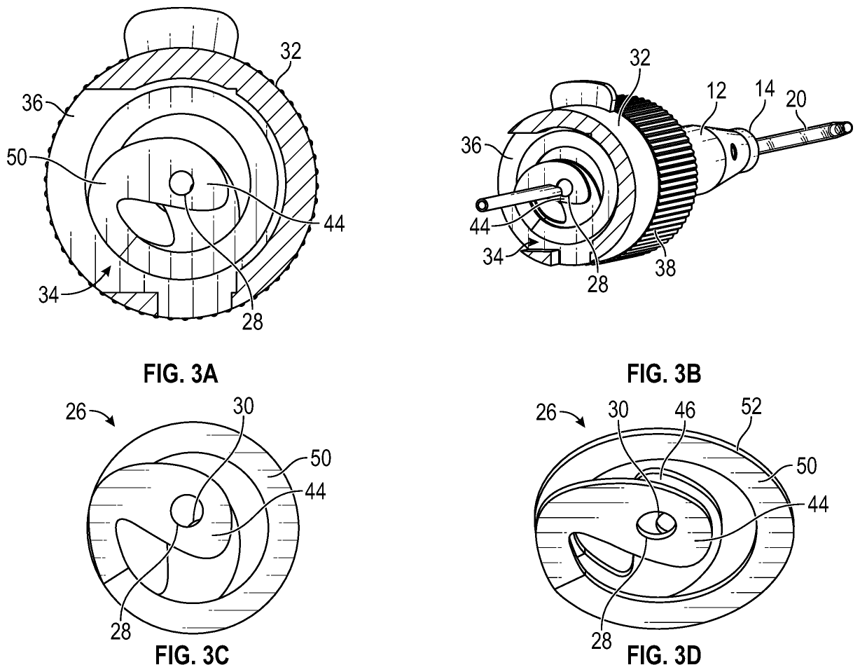

In some embodiments, the introducer needle 24 may be moved proximally to a retracted position, such as illustrated, for example, in FIGS. 1B and 1D. In some embodiments, in response to movement of the introducer needle 24 proximally to a retracted position, the introducer needle 24 may be removed from the first hole 28 and the second hole 30 and the compressible element 26 may expand. In some embodiments, in response to the compressible element 26 expanding, the first hole 28 and the second hole 30 may misalign or move apart, as illustrated, for example, in FIG. 3A, shielding the introducer needle 24 from distal movement beyond the compressible element 26, thereby reducing a risk of escape of the introducer needle 24 from the housing 32 and subsequent needle stick injury.

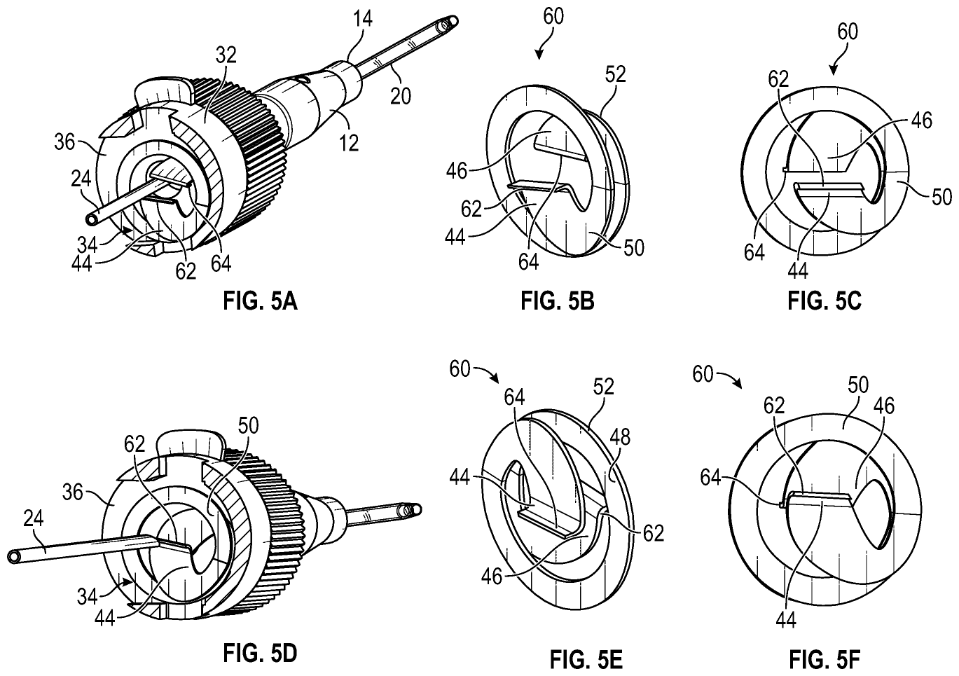

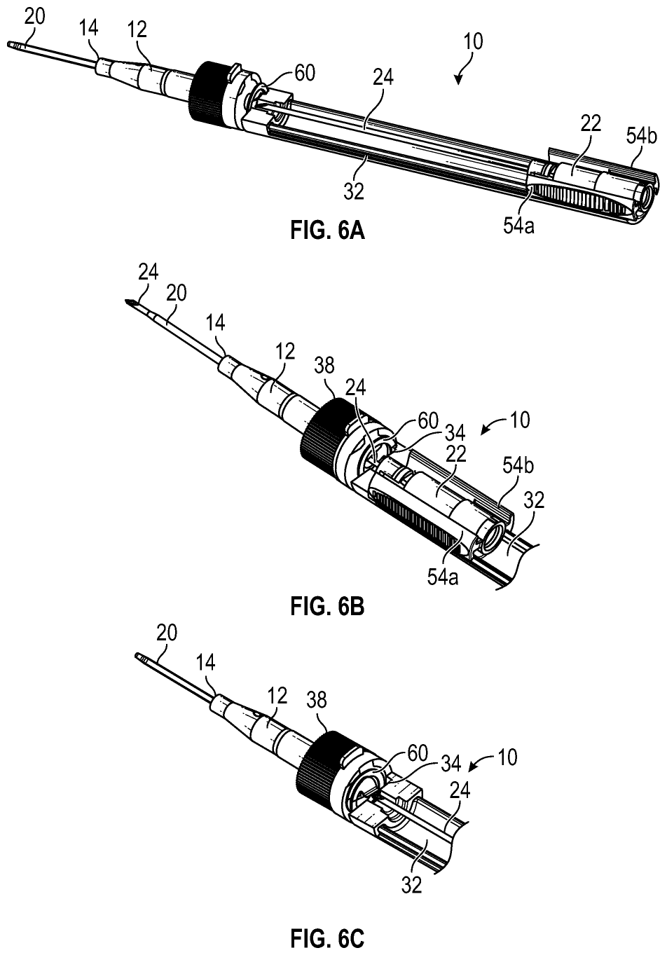

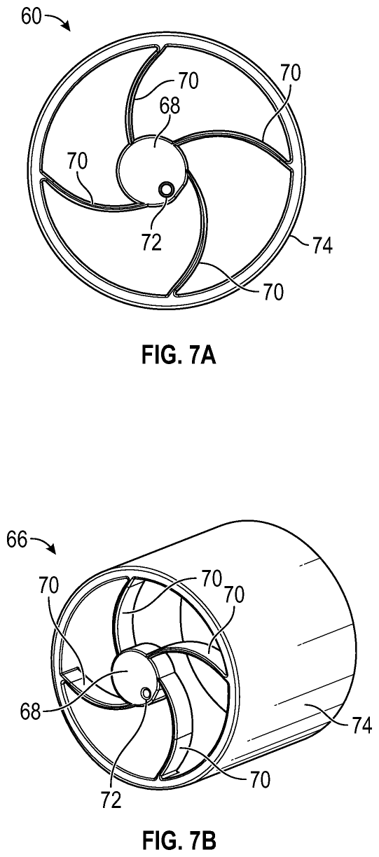

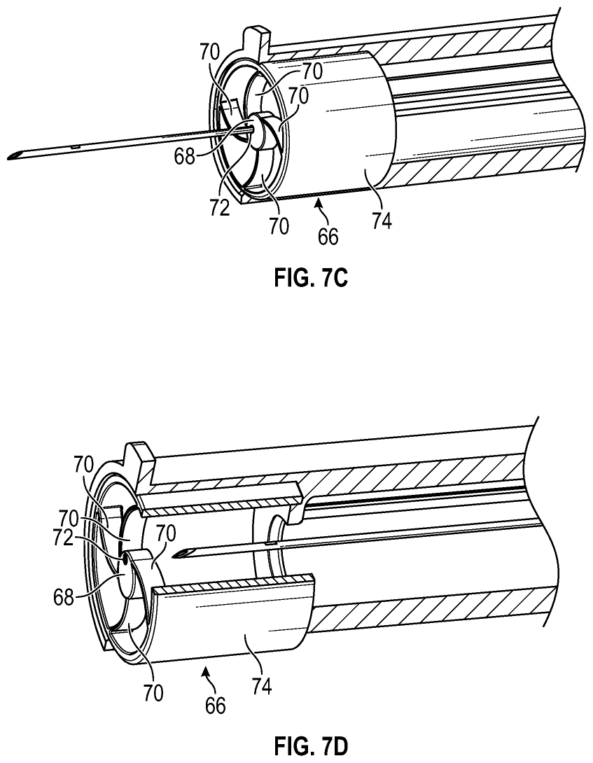

In some embodiments, the compressible element 26 (or the compressible element 60 of FIGS. 5-6 or the needle shield element 66 of FIG. 7) may be configured to block or shield the introducer needle 24 in a passive manner. Thus, the catheter system 10 may block or shield the introducer needle 24 automatically in response to proximal retraction of the introducer needle 24 from the advanced position to the retracted position. In these embodiments, the compressible element 26 may provide passive safety and also reduce blood splatter due to a reduced speed of the introducer needle 24 during the proximal retraction of the introducer needle 24 that is manual. In some embodiments, the needle hub 22 may travel from a first position in which the introducer needle 24 is advanced to a second position in which the introducer needle 24 is retracted. In some embodiments, in response to the needle hub 22 moving from the first position to the second position, the compressible element will expand to protect the introducer needle 24 within the housing 32.

In some embodiments, the catheter system 10 may include a housing 32, which may include a barrel. In some embodiments, the housing 32 may include a pocket 34 and an insertion slot 36 transverse to a longitudinal axis of the housing 32 and providing access to the insertion slot 36. In some embodiments, a distal end 38 of the housing 32 may be coupled to the proximal end 16 of the catheter hub 12. In some embodiments, the compressible element 26 may be disposed within the pocket 34. In some embodiments, the compressible element 26 may be configured to compress in order to fit through the insertion slot 36 and into the pocket 34 during assembly of the catheter system 10. In some embodiments, the compressible element 26 may be constructed of metal, plastic, or another suitable material.

In some embodiments, the housing 32 may include an elongated slot 42 aligned with the longitudinal axis of the housing 32. In some embodiments, the distal end 38 of the housing 32 may be coupled to the proximal end 16 of the catheter hub 12. In some embodiments, the distal end 38 of the housing 32 may include a luer adapter or another type of connector configured to form a fluid tight seal with the proximal end 16 of the catheter hub 12. In some embodiments, the luer adapter may include a small protrusion that locks rotation of the catheter hub 12 and ensures a bevel of the introducer needle 24 is facing upwards. In some embodiments, the distal end 38 of the housing 32 may include a male luer adapter.

In some embodiments, the needle hub 22 may extend through the elongated slot 42 and may be configured to move along the elongated slot 42 to move the introducer needle 24 from the advanced position to the retracted position. In some embodiments, the housing 32 may include a proximal end 40, and the needle hub 22 may slide along the elongated slot 42 towards the proximal end 40 to the retracted position in order to retract the introducer needle 24. In some embodiments, the needle hub 22 may include one or more grip elements 43 configured for friction with a finger of a user to facilitate sliding of the needle hub 22 along the elongated slot 42.

In some embodiments, a proximal end of the elongated slot 42 may include a retainer snap 45. In some embodiments, the retainer snap 45 may prevent the needle hub 22 from exiting the elongated slot 42 while the retracting the introducer needle 24 or moving the introducer needle 24 from the advanced position to the retracted position. In some embodiments, the retainer snap 45 may include two opposing protrusions, which may be spaced apart. In some embodiments, the retainer snap 45 may facilitate placement of the needle hub 22 within the elongated slot 42 during assembly of the catheter system 10.

In some embodiments, the compressible element 26 may include a coil shape, as illustrated, for example, in FIGS. 2C-2E and 3C-3D. In some embodiments, the first hole 28 may be disposed in a first end 44 of the coil shape and the second hole 30 may be disposed in a second end 46 of the coil shape. In some embodiments, the coil shape may be flattened, which may facilitate insertion of the compressible element 26 through the insertion slot 36. In further detail, in some embodiments, the coil shape may include a distally-facing surface 48, a proximally-facing surface 50, and an edge 52 transverse to the distally-facing surface 48 and the proximally-facing surface 50. In other embodiments, the coil shape may not be flattened. In some embodiments, the coil shape may form a ring, which may include an outer edge of the compressible element 26. In some embodiments, the first end 44 of the coil shape and the second end 46 of the coil shape may be positioned inwardly from the ring or the outer edge.

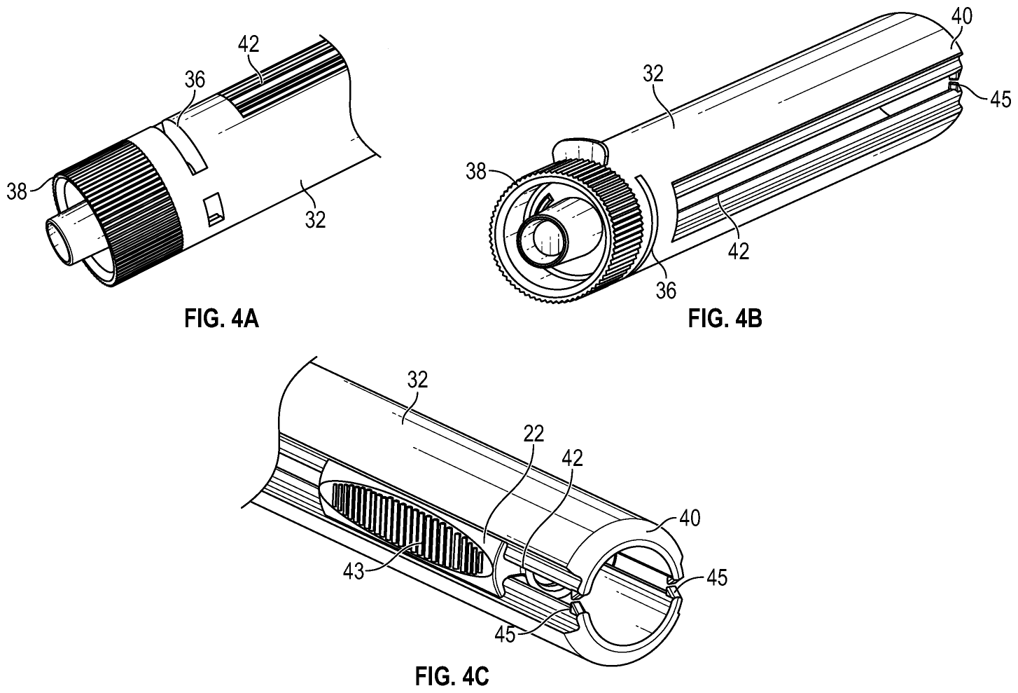

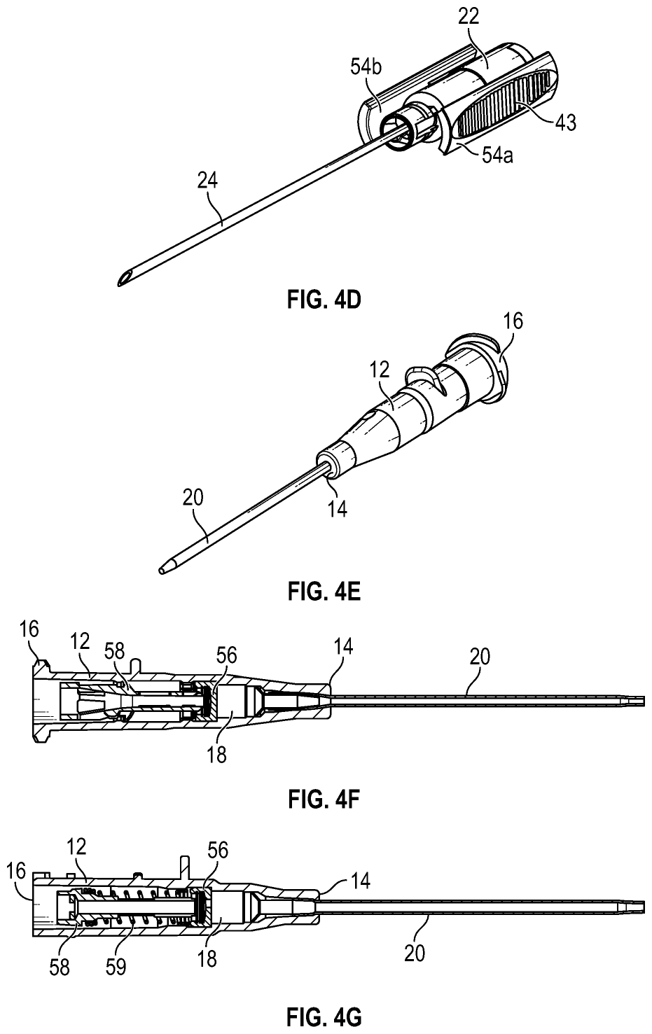

Referring now to FIG. 4, in some embodiments, the elongated slot 42 may be a first elongated slot 42a, and the housing 32 may include a second elongated slot 42b aligned with the longitudinal axis of the housing 32. In some embodiments, the second elongated slot 42b may be similar to the first elongated slot 42a in terms of one or more features and/or operation. In some embodiments, the needle hub 22 may extend through the first elongated slot 42a and the second elongated slot 42b. In some embodiments, the needle hub 22 may be configured to move along the first elongated slot 42a and the second elongated slot 42b to move the introducer needle 24 from the advanced position to the retracted position.

As illustrated in FIG. 4D, in some embodiments, the needle hub 22 may include one or more elongated slider elements 54, which may extend across the elongated slot 42. In some embodiments, the grip elements 43 may be disposed on the elongated slider elements 54. In some embodiments, the elongated slider elements 54 may be positioned outwardly from a generally cylindrical feature that is assembled concentrically with the introducer needle 24 and/or includes a vent plug. In some embodiments, the needle hub 22 may include two elongated slider elements 54a, 54b, which may be opposing and configured to extend across the first elongated slot 42a and the second elongated slot 42b, respectively. In some embodiments, the elongated slider elements 54a, 54b may be disposed on a left and right side, respectively, of the catheter system, which may facilitate sliding with a thumb and index finger, for example. As illustrated in FIG. 4F, in some embodiments, the catheter hub 12 may include blood control, such as a septum 56 and/or a septum actuator 58.

As illustrated, for example, in FIG. 4G, in some embodiments, a spring 59 may be disposed within the catheter hub 12 and coupled to the septum actuator 58. In some embodiments, in response to coupling of a medical device, such as a male luer (not shown), to the proximal end 16 of the catheter hub, the septum actuator 58 may be urged distally from an initial position by the medical device and may penetrate a slit of the septum 56. In some embodiments, the spring 59 may facilitate return of the septum actuator 58 to the initial position proximal to the septum 56 in response to removal of the medical device from the proximal end 16 of the catheter hub 12. Thus, in some embodiments, a catheter assembly may be multi-use with the septum actuator 58 coupled to the spring 59, facilitating opening and closing of the septum 56. In other embodiments, the catheter assembly may be single-use having a particular septum actuator 58 that keeps the septum 56 open even after the medical device is removed (see FIG. 4F, for example). It is understood that in some embodiments, the catheter assembly, including the catheter 20 and/or the catheter hub 12, may include a peripheral intravenous catheter assembly. In other embodiments, the catheter assembly may include a midline catheter assembly, a peripherally-inserted central catheter assembly, or another suitable type of catheter assembly.

Referring now to FIG. 5, in some embodiments, the catheter system 10 of FIGS. 1-4 may include a compressible element 60 instead of the compressible element 26. In some embodiments, the compressible element 60 may be similar or identical to the compressible element 26 in terms of one or more features and/or operation. In some embodiments, the compressible element 60 may be constructed of metal, plastic, or another suitable material. In some embodiments, the compressible element 60 may include a first flange 62 and a second flange 64. In some embodiments, the introducer needle 24 may extend between the first flange 62 and the second flange 64 when the introducer needle 24 is in the advanced position. In some embodiments, when the introducer needle 24 is in the advanced position, the first flange 62 and the second flange 64 may press against the introducer needle 24.

In some embodiments, in response to movement of the introducer needle 24 proximally to the retracted position, the introducer needle 24 may not contact the first flange 62 and the second flange 64 and the first flange 62 may overlap the second flange 64, shielding the introducer needle 24 from distal movement beyond the compressible element 60. In these embodiments, when the introducer needle 24 is in the retracted position, the first flange 62 and the second flange 64 may move towards each other such that the introducer needle 24 cannot pass therethrough. In some embodiments, the compressible element 60 may include the coil shape, which may be flattened and/or form the ring. In some embodiments, the first flange 62 and the second flange 64 may be positioned inwardly from the ring.

Referring now to FIG. 7, in some embodiments, the catheter system 10 of FIGS. 1-4 may include a needle shield element 66 instead of the compressible element 26. In some embodiments, the needle shield element 66 may include a movable center 68 and multiple flexible arms 70 extending outwardly from the movable center 68. In some embodiments, the movable center 68 may include an aperture 72 therethrough. In some embodiments, the aperture 72 may be disposed on an eccentric side of the movable center 68 or off-center within the movable center 68. In some embodiments, the introducer needle 24 may extend through the aperture 72 when the introducer needle 24 is in the advanced position biasing the movable center 68 in a first position, illustrated, for example, in FIG. 7E. In some embodiments, the introducer needle 24 extend through the aperture 72 may cause the flexible arms 70 to move in a twisting motion to bias the movable center 68 in the first position. In some embodiments, the needle shield element 66 and/or the flexible arms 70 may be constructed of plastic or another suitable material.

In some embodiments, in response to movement of the introducer needle 24 proximally from the advanced position to the retracted position, the introducer needle 24 may be removed from the aperture 72 and the movable center 68 may move to a second position, illustrated, for example, in FIG. 7F, in which the aperture 72 is offset from or misaligned with a longitudinal axis of the introducer needle 24, shielding the introducer needle 24 from distal movement beyond the needle shield element 66.

In some embodiments, the needle shield element 66 may be disposed within the housing 32. In some embodiments, the flexible arms 70 may extend from the movable center 68 to an outer ring 74 of the needle shield element 66. In some embodiments, the outer ring 74 may be fitted within the housing 32, which may include the barrel, and secured therein. In other embodiments, the flexible arms 70 may extend from the movable center 68 to an outer ring 74 of the needle shield element 66 directly to an inner surface of the housing 32 to which the flexible arms 70 may be secured.

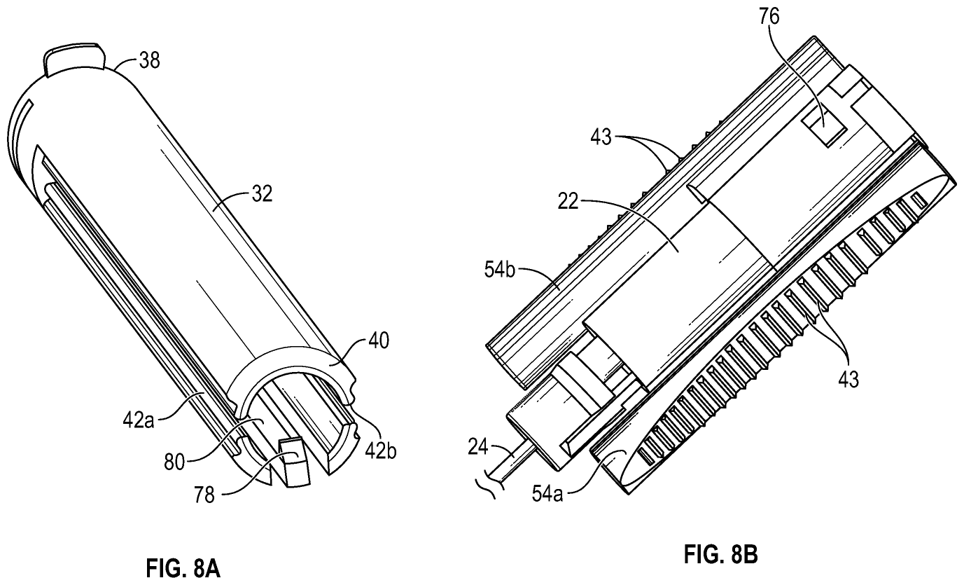

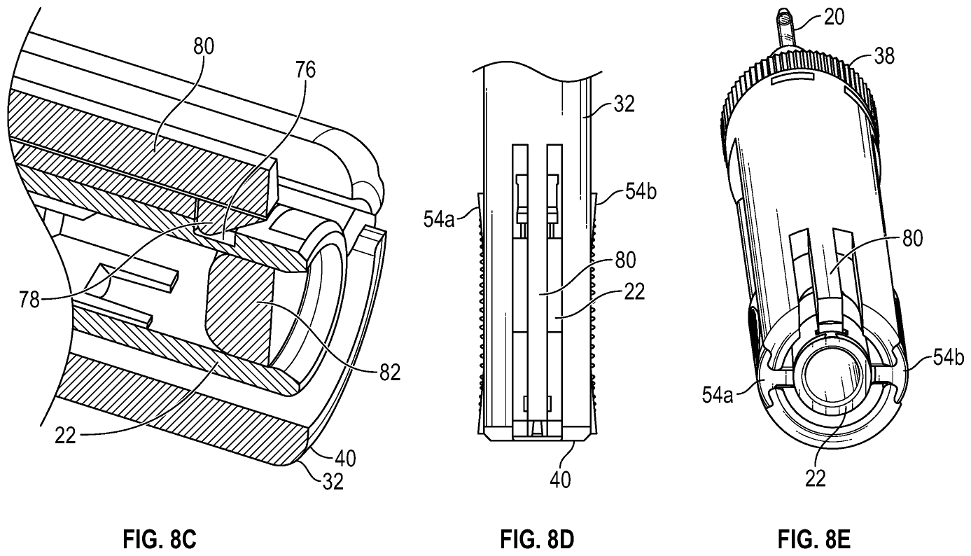

Referring now to FIGS. 8A-8E, in some embodiments, the needle hub 22 may include a hub lock slot 76. In some embodiments, the proximal end 40 of the housing 32 may include a rib 78 configured to lock within the hub lock slot 76 in response to the introducer needle 24 being moved from the advanced position to the retracted position. In some embodiments, the rib 78 may be disposed on a flexible arm 80 of the housing 32, which may facilitate movement of the rib 78 along an outer surface of the needle hub 22 until the rib 78 is disposed within the hub lock slot 76. In some embodiments, the needle hub 22 may include a vent plug 82.

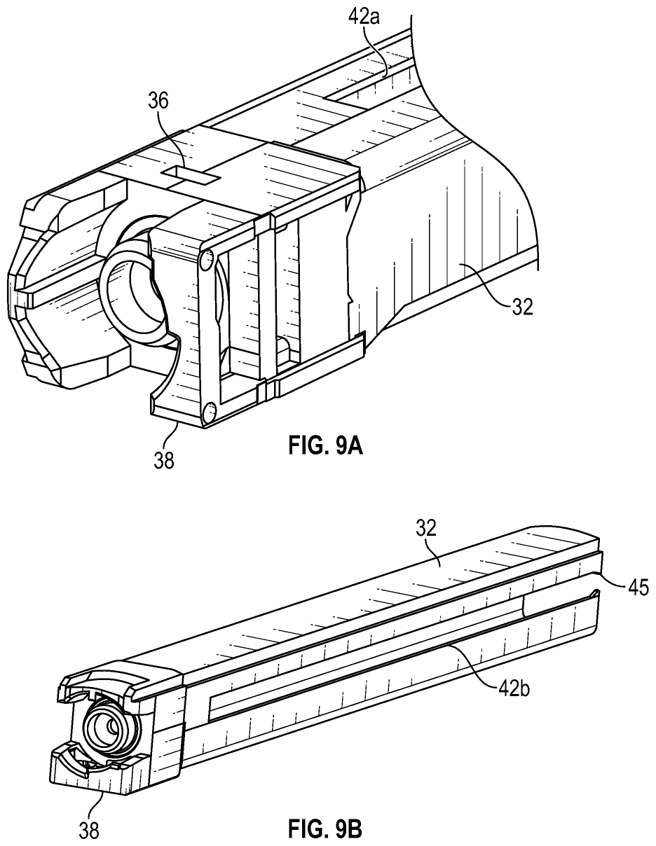

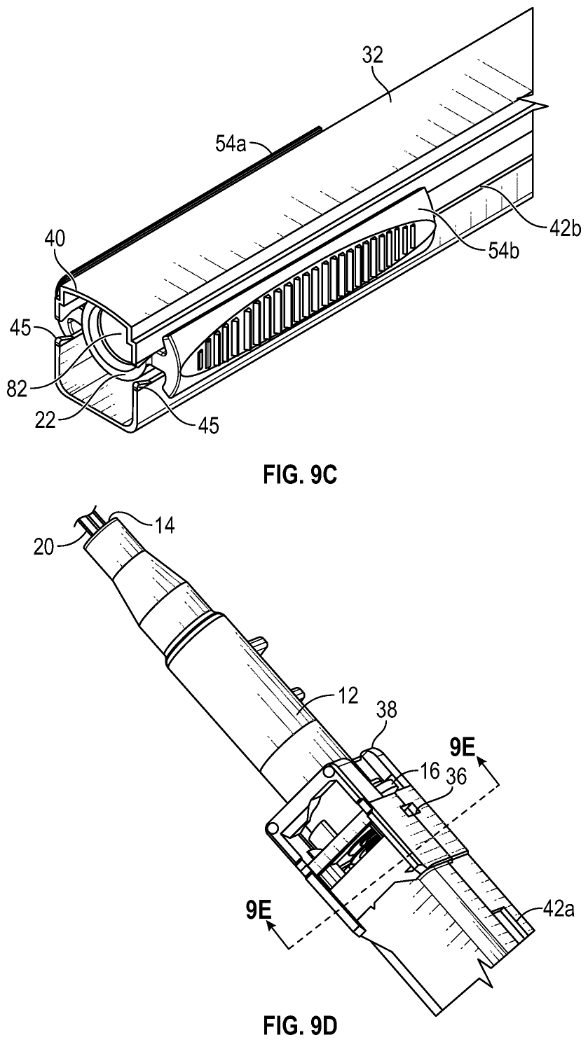

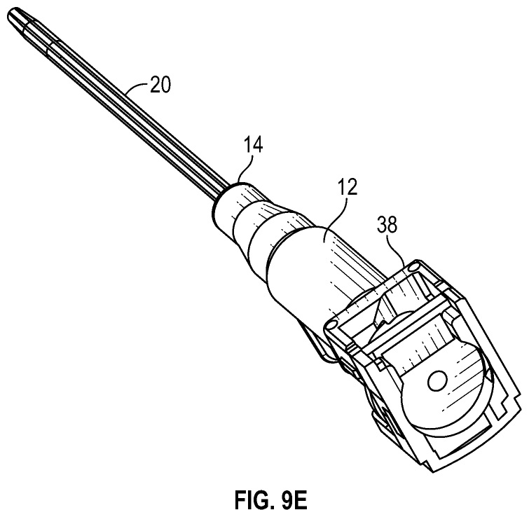

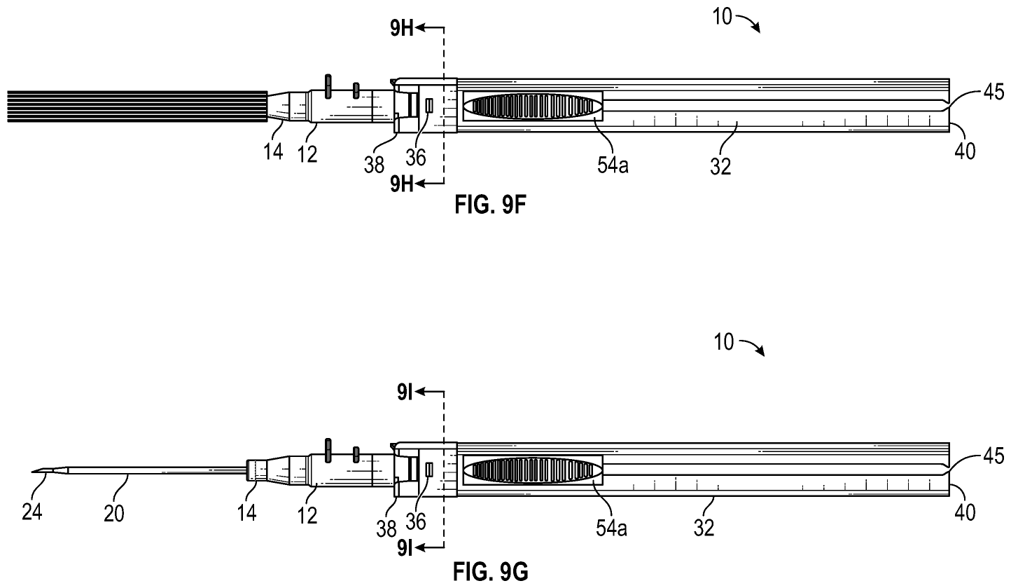

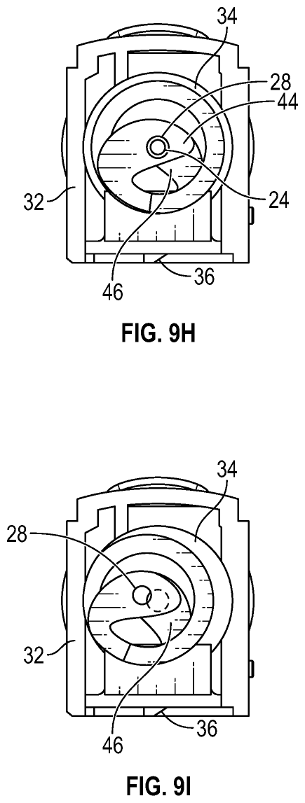

Referring now to FIG. 9, in some embodiments, a shape of the housing 32 of the catheter system 10 may vary. For example, the housing 32 may be cylindrical or rectangular or another suitable shape. FIG. 9F illustrates a cover that may be placed over the catheter 20 and the introducer needle 24 prior to use of the catheter system 10.

All examples and conditional language recited herein are intended for pedagogical objects to aid the reader in understanding the invention and the concepts contributed by the inventor to furthering the art and are to be construed as being without limitation to such specifically recited examples and conditions. Although embodiments of the present inventions have been described in detail, it should be understood that the various changes, substitutions, and alterations could be made hereto without departing from the spirit and scope of the invention.

What is claimed:

1. A catheter system, comprising:
a catheter hub, comprising a distal end, a proximal end, and a lumen extending through the distal end and the proximal end;
a catheter extending from the distal end of the catheter hub;
a needle hub;
an introducer needle extending distally from the needle hub, wherein the introducer needle extends through the catheter when the introducer needle is in an advanced position,
a compressible element comprising a first hole and a second hole, wherein the introducer needle extends through the first hole and the second hole when the introducer needle is in the advanced position, wherein in response to movement of the introducer needle proximally to a retracted position, the introducer needle is removed from the first hole and the second hole and the compressible element expands, wherein in response to the compressible element expanding, the first hole and the second hole misalign, shielding the introducer needle from distal movement beyond the compressible element.

2. The catheter system of claim 1, further comprising a housing, wherein the housing comprises a pocket and an insertion slot transverse to a longitudinal axis of the housing and providing access to the insertion slot, wherein a distal end of the housing is coupled to the proximal end of the catheter hub, wherein the compressible element is disposed within the pocket, wherein the compressible element is configured to compress in order to fit through the insertion slot and into the pocket.

3. The catheter system of claim 1, further comprising a housing comprising an elongated slot aligned with a longitudinal axis of the housing, wherein a distal end of the housing is coupled to the proximal end of the catheter hub, wherein the needle hub extends through the elongated slot and is configured to move along the elongated slot to move the introducer needle from the advanced position to the retracted position.

4. The catheter system of claim 3, wherein the elongated slot is a first elongated slot, wherein the housing comprises a second elongated slot aligned with the longitudinal axis of the housing, wherein the needle hub extends through the first elongated slot and is configured to move along the first elongated slot and the second elongated slot to move the introducer needle from the advanced position to the retracted position.

5. The catheter system of claim 3, wherein a proximal end of the elongated slot comprises a retainer snap.

6. The catheter system of claim 1, wherein the compressible element comprises a coil shape, wherein the first hole is disposed in a first end of the coil shape and the second hole is disposed in a second end of the coil shape.

7. The catheter system of claim 6, wherein the coil shape is flattened and forms a ring, wherein the first end and the second end are positioned inwardly from the ring.

8. The catheter system of claim 1, wherein a lumen of the catheter hub comprises a blood-control septum, a septum actuator, and a spring coupled to the septum actuator, wherein the spring is configured to return the septum actuator to an initial position proximal to the septum in response to removal of the medical device from the proximal end of the catheter hub.

9. A catheter system, comprising:
a catheter hub, comprising a distal end, a proximal end, and a lumen extending through the distal end and the proximal end;
a catheter extending from the distal end of the catheter hub;
a needle hub;
an introducer needle extending distally from the needle hub, wherein the introducer needle extends through the catheter when the introducer needle is in an advanced position,
a compressible element comprising a first flange and a second flange, wherein the introducer needle extends between the first flange and the second flange when the introducer needle is in the advanced position, wherein in response to movement of the introducer needle proximally to a retracted position, the introducer needle does not contact the first flange and the second flange and the first flange overlaps the second flange, shielding the introducer needle from distal movement beyond the compressible element.

10. The catheter system of claim 9, further comprising a housing, wherein the housing comprises a pocket and an insertion slot transverse to a longitudinal axis of the housing and providing access to the insertion slot, wherein a distal end of the housing is coupled to the proximal end of the catheter hub, wherein the compressible element is disposed within the pocket, wherein the compressible element is configured to compress in order to fit through the insertion slot and into the pocket.

11. The catheter system of claim 9, further comprising a housing comprising an elongated slot aligned with a longitudinal axis of the housing, wherein a distal end of the housing is coupled to the proximal end of the catheter hub, wherein the needle hub extends through the elongated slot and is configured to move along the elongated slot to move the introducer needle from the advanced position to the retracted position.

12. The catheter system of claim 11, wherein the elongated slot is a first elongated slot, wherein the housing comprises a second elongated slot aligned with the longitudinal axis of the housing, wherein the needle hub extends through the first elongated slot and is configured to move along the first elongated slot and the second elongated slot to move the introducer needle from the advanced position to the retracted position.

13. The catheter system of claim 11, wherein a proximal end of the elongated slot comprises a retainer snap.

14. The catheter system of claim 9, wherein the compressible element comprises a coil shape, wherein the first flange is disposed at a first end of the coil shape and the second flange is disposed at a second end of the coil shape.

15. The catheter system of claim 9, wherein a lumen of the catheter hub comprises a blood-control septum, a septum actuator, and a spring coupled to the septum actuator, wherein the spring is configured to return the septum actuator to an initial position proximal to the septum in response to removal of the medical device from the proximal end of the catheter hub.

16. A catheter system, comprising:
a catheter hub, comprising a distal end, a proximal end, and a lumen extending through the distal end and the proximal end;
a catheter extending from the distal end of the catheter hub;
a needle hub;
an introducer needle extending distally from the needle hub, wherein the introducer needle extends through the catheter when the introducer needle is in an advanced position,
a needle shield element, comprising a movable center and a plurality of flexible arms extending outwardly from the movable center, wherein the movable center comprises an aperture therethrough, wherein the introducer needle extends through the aperture when the introducer needle is in the advanced position biasing the movable center in a first position, wherein in response to movement of the introducer needle proximally to a retracted position, the introducer needle is removed from the aperture and the movable center moves to a second position in which the aperture is offset from a longitudinal axis of the introducer needle, shielding the introducer needle from distal movement beyond the needle shield element.

17. The catheter system of claim 16, further comprising a housing comprising an elongated slot aligned with a longitudinal axis of the housing, wherein a distal end of the housing is coupled to the proximal end of the catheter hub, wherein the needle hub extends through the elongated slot and is configured to move along the elongated slot to move the introducer needle from the advanced position to the retracted position.

18. The catheter system of claim 17, wherein the elongated slot is a first elongated slot, wherein the housing comprises a second elongated slot aligned with the longitudinal axis of the housing, wherein the needle hub extends through the first elongated slot and is configured to move along the first elongated slot and the second elongated slot to move the introducer needle from the advanced position to the retracted position.

19. The catheter system of claim 17, wherein a proximal end of the elongated slot comprises a retainer snap.

20. The catheter system of claim 17, wherein the needle shield element is disposed within the housing.

21. The catheter system of claim 17, wherein the needle hub comprises a hub lock slot, wherein a proximal end of the housing comprises a rib configured to lock within the hub lock slot in response to the introducer needle being moved from the advanced position to the retracted position.

22. The catheter system of claim 16, wherein a lumen of the catheter hub comprises a blood-control septum, a septum actuator, and a spring coupled to the septum actuator, wherein the spring is configured to return the septum actuator to an initial position proximal to the septum in response to removal of the medical device from the proximal end of the catheter hub.
